(19) United States
(12) Patent Application Publication
(10) Pub. No.: US 2003/0171812 A1
(43) Pub. Date: Sep. 11, 2003

(54) Minimally invasive modular support implant device and method

(76) Inventors: Ilan Grunberg, Haifa (IL); Nissim Ohana, Raanana (IL); Asaf Ben-Arye, Zichron Yaakov (IL)

Correspondence Address: SALTAMAR INNOVATIONS, 30 FERN LANE, SOUTH PORTLAND, ME, 04106, US

(21) Appl. No.: 10/036,564

(22) Filed: Dec. 31, 2001

(51) Int. Cl. A61F 2/44; A61B 17/80

(52) U.S. Cl. 623/17.11; 606/71

(57) ABSTRACT

device and method are disclosed of a plate for use in conjunction with at least another one of a plurality of other plates in a modular reconstructing and supporting assembly for reconstructing and supporting a diseased or fractured bone or within a space previously occupied by a diseased intervertebral disc of a patient. The plate is sized small enough to be suitable for separate insertion into the bone or the space, preferably through a canule, and arrangement with the other plates adjacently one on top of the other to construct scaffolding, so as to provide a supporting prosthesis.

In another preferred embodiment the plate has at least two substantially opposite aspects with interlocking features designed to facilitate interlocking of adjacent plates so as to prevent or restrain their sliding off each other.

### FIELD OF THE INVENTION

The present invention relates to orthopedic implants. More particularly it relates to implants providing minimally invasivemodular support implant device and method.

### BACKGROUND OF THE INVENTION

The spinal column serves as the support structure of the body, rendering the body its posture. Yet age, diseases and traumas hamper its completeness, and health, causing structural failures such as vertebral fractures, disc hernias, degenerative disk diseases, etc., resulting in pain and spinal instability, and even paralysis.

The adult vertebral column includes 26 vertebras (7 cervical, 12 thoracic, 5 lumbar, 1 sacrum and 1 coccyx) separated by intervertebral fibrocartilage discs.

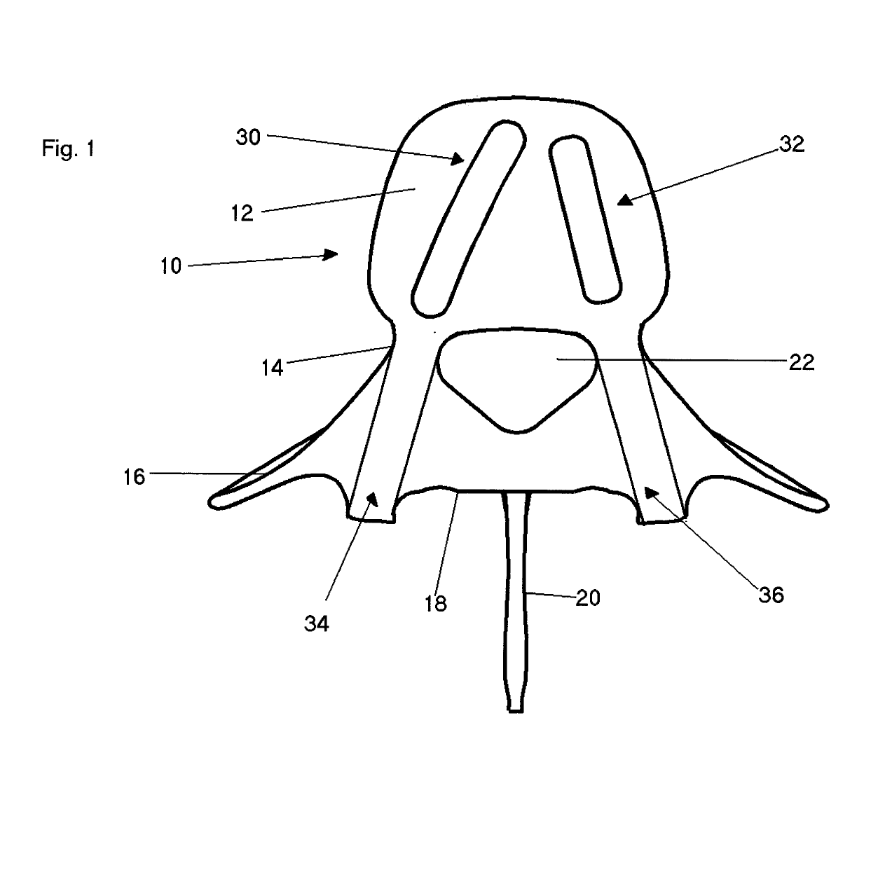

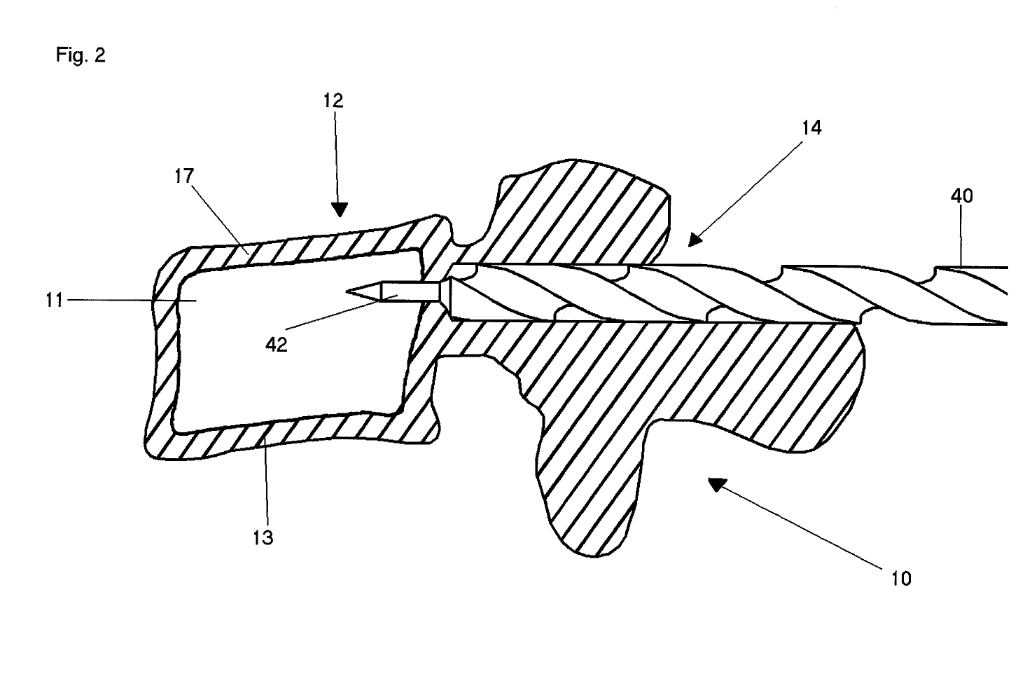

A typical vertebra 10 consists of two essential parts—an anterior segment, comprising the body 12, and a posterior part, comprising the vertebral or neural arch The vertebral arch consists of a pair of pedicles 14 and a pair of laminae 18, and supports seven processes—four articular, two transverse 16, and one spinous 20 (see FIG. 1). The body and the vertebral arch define a foramen, known as the vertebral foramen 22. It should be noted that the vertebras' structure differs slightly according to the position on the spinal column (i.e. cervical, thoracic, lumbar).

Among various vertebral column disorders the typical ones include traumatic damages such as compression fractures, degenerative disc disease, disc hernias (ruptured or protruded disc), scoliosis (lateral bending of the vertebral column), kyphosis (exaggerated thoracic curvature), lordosis (exaggerated lumbar curvature), spina bifidia (congenital incompletion of the closure of the vertebral column).

Various fixation, replacement and reconstructive solutions—both intravertebral and intervertebral were introduced in the past, some of which are mentioned hereinafter.

For example, U.S. Pat. No. 6,019,793 (Perren et al.), titled SURGICAL PROSTHETIC DEVICE, disclosed a surgical prosthetic device that is adapted for placement between two adjoining vertebrae for total or partial replacement of the disk from therebetween. The device has two plates with interior surfaces facing each other and being held at a distance by connecting means and exterior surfaces for contacting the end plates of the two adjoining vertebrae. The connecting means is made of a shape-memory alloy so that it is delivered to its destination cramped within a delivering tool and deploys once freed in position.

U.S. Pat. No. 5,423,816 (Lin) titled INTERVERTEBRAL LOCKING DEVICE disclosed an intervertebral locking device comprising one spiral elastic body, two bracing mounts and two sets of locking members. The two bracing mounts are fastened respectively to both ends of the spiral elastic body. The two sets of locking members are fastened respectively with the two bracing mounts such that each set of the locking members is anchored in one of the two vertebrae adjacent to a vertebra under treatment. The spiral elastic body and the vertebra under treatment evince similar elastic qualities, i.e. similar deflection characteristics. A plurality of bone grafts affinitive to the vertebra under treatment is deposited in the chambers of the spiral elastic body and in the spaces surrounding the spiral elastic body.

U.S. Pat. No. 5,423,817 (Lin) titled INTERVERTEBRAL FUSING DEVICE, teaches an intervertebral fusing device having a spring body portion interconnecting a first spiral ring mount and a second spiral ring mount. Each spiral ring mount has a spiralling projection on the outer surface. The spring body portion is defined by a plurality of spiral loops. The plurality of spiral loops and spiralling projection of the spiral ring mounts have a constant pitch. A mount cover and a head member are threaded into an internally threaded portion of a respective spiral ring mount thereby forming a chamber in which bone grafts affinitive to the cells and tissues of a vertebra may be housed. The spring body portion is similar in elasticity to the vertebra.

U.S. Pat. No. 5,306,310 (Siebels), titled VERTEBRAL PROSTHESIS, disclosed a prosthesis as a vertebral replacement element consisting of two helical strands, which may be screwed together to form a tubular structure. The implant is inserted between vertebrae and then slightly unscrewed until the desired height is reached. The helical strands consist of carbon fiber reinforced composite material.

U.S. Pat. No. 6,033,406 (Mathews) titled METHOD FOR SUBCUTANEOUS SUPRAFASCIAL PEDICULAR INTERNAL FIXATION disclosed a method for internal fixation of vertebra of the spine to facilitate graft fusion includes steps for excising the nucleus of an affected disc, preparing a bone graft, instrumenting the vertebrae for fixation, and introducing the bone graft into the resected nuclear space. Disc resection is conducted through two portals through the annulus, with one portal supporting resection instruments and the other supporting a viewing device. The fixation hardware is inserted through small incisions aligned with each pedicle to be instrumented. The hardware includes bone screws, fixation plates, engagement nuts, and linking members. In an important aspect of the method, the fixation plates, engagement nuts and linking members are supported suprafascially but subcutaneously so that the fascia and muscle tissue are not damaged. The bone screw is configured to support the fixation hardware above the fascia. In a further aspect of the invention, a three-component dilator system is provided for use during the bone screw implantation steps of the method.

Generally, these described methods and devices are very invasive and involve massive surgical involvement.

Minimally invasive system is described in U.S. Pat. No. 6,248,110 (Reiley et al.) titled SYSTEMS AND METHODS FOR TREATING FRACTURED OR DISEASED BONE USING EXPANDABLE BODIES. Systems and methods are disclosed for treating fractured or diseased bone by deploying more than a single therapeutic tool into the bone. In one arrangement, the systems and methods deploy an expandable body in association with a bone cement nozzle into the bone, such that both occupy the bone interior at the same time. In another arrangement, the systems and methods deploy multiple expandable bodies, which occupy the bone interior volume simultaneously. Expansion of the bodies form cavity or cavities in cancellous bone in the interior bone volume. Use of expandable balloon is taught, which serves for reconstruction of collapsed bone. In order to fill the space created and provide stabilization to the bone insertion of polymethylmethacrylate cement is required, which dries and stiffens. The above-mentioned fixation and support solutions (and others) all introduce mechanical structures to gain support of fixation. All devices are surgically placed in the desired position. Some of them require a major surgical operation involving major invasive actions. The problem with polymethylmethacrylate (PMMA) cement is that it is not suitable for insertion in young people because it tends to loosen hence the fixation is jeopardized. In addition it may involve side effects such as spinal cord injuries, radiculopathies, and cement leakage. Furthermore the cement is hard to control and maintain during insertion because of its fluidic nature and consistency.

### BRIEF DESCRIPTION OF THE INVENTION

It is the purpose of the present invention to provide a minimally invasive method and system for reconstructing and supporting a fractured or diseased bone, preferably a fractured or diseased vertebra. In an alternative embodiment of the present invention the method and system disclsed herein are aimed at providing support within a space previously occupied by a diseased intervertebral disc, that has been completely or partially removed.

It is therefore provided, in accordance with a preferred embodiment of the present invention, a plate for use in conjunction with at least another one of a plurality of other plates in a modular reconstructing and supporting assembly for reconstructing and supporting a diseased or fractured bone or within a space previously occupied by a diseased intervertebral disc of a patient, the plate sized small enough to be suitable for separate insertion into the bone or the space, and arrangement with the other plates adjacently one on top of the other to construct scaffolding, so as to provide a supporting prosthesis.

Furthermore, in accordance with a preferred embodiment of the present invention, the plate has at least two substantially opposite aspects with interlocking features designed to facilitate interlocking of adjacent plates so as to prevent or restrain their relative movement. In an optional preferred embodiment, the opposite aspects of the plate are inclined with respect to each other. In the preferred embodiment, one aspect is provided with at least one longitudinal protrusion and the opposite aspect is provided with at least one corresponding longitudinal recess designed to accommodate a longitudinal protrusion of an adjacent plate. Alternatively or in combination, one aspect is provided with at least one lateral protrusion and the opposite aspect is provided with at least one corresponding longitudinal recess designed to accommodate a lateral protrusion of an adjacent plate. Similarly combinations that allow for a single aspect to have a longitudinal protrusion and lateral recess, or similar interlocking combinations are also contemplated.

succinctly stated, the preferred interlocking embodiment of the present invention contemplates interlocking features that include at least one recess on one aspect and at least one corresponding projection on the other aspect, so that the projection of one plate is accommodated in the recess of an adjacent plate.

Furthermore, in the preferred embodiment of the present invention, the recess ends with a rim aimed at retaining the projection of an adjacent plate preventing or restraining relative displacement of adjacent plates. The rim is provided partially, allowing leveled sliding in of the projection of the adjacent plate.

Furthermore, in accordance with an embodiment of the present invention, the plate is curved.

The plate may also be provided with at least one tapered end, so as to facilitate guiding of the the plate through and positioning it between two adjacent plates, whilst separating them and sliding therebetween. Conveniently, the tapered end may be in the form of a wedge.

In the preferred embodiment of the present invention, the plate is made from or coated with biocompatible material. Preferably, the plate is made from material selected from metals such as titanium, steel 316, processed foil, hydroxyapatite, or material coated with hydroxyapetite, plastics, silicon, composite materials, such as carbon-fiber. More preferably, the plate is covered with substance encouraging bone growth, such as bone morphogenic protein. Alternatively or in combination, the plate is covered with medication substances, such as antibiotics, or slow releasing medication, such as chemotherapic substences. The plate may also be coated with lubrucanting material to facilitate sliding of the plates into position. The plate may also be coated with coating materias that sublime or react to form a solid conglomerate. It will be understood that such coatings and materials may be used in combination.

In the preferred embodiment of the present invention, the plate is made from non-ferrous material.

Furthermore, in accordance with the preferred embodiment of the present invention, the plate is disc-shaped.

Preferably, the plate is provided with a groove to enhance holding of the plate by a tool. The plate may further provided with a protruding pin serving for holding the plate by a delivering tool so as to ensure safe guiding in into position. The plate may also be provided with rough surfaces.

In another aspect of the present invention, there is provided a plate for use in conjunction with at least another one of a plurality of other plates in a modular reconstructing and supporting assembly for reconstructing and supporting a diseased or fractured bone or within a space previously occupied by a diseased intervertebral disc of a patient, the plate sized small enough to be suitable for separate insertion into the bone or the space and arrangement with the other plates adjacently to construct scaffolding, so as to provide a supporting prosthesis.

Furthermore, in accordance with another aspect of the present invention, there is provided a modular reconstructing and supporting assembly for reconstructing and supporting a diseased or fractured bone or within a space previously occupied by a diseased intervertebral disc, the assembly comprising a plurality of plates each adapted to be separately inserted into the bone and arranged one on top of the other within the bone or the space to construct scaffolding, thus providing a supporting prosthesis.

The present invention further provides a method for reconstructing and supporting within a diseased or fractured bone or within a space previously occupied by a diseased intervertebral disc the method comprising:

providing a plurality of plates adapted to be separately inserted into the bone and arranged adjacently one on top of the other within the bone or space to construct scaffolding for providing support;

providing a delivery tool having a low profile for delivering plates through a small incision in the skin of the patient and into the bone or disc;

delivering at least one plate separately into the bone;

arranging the plates adjacently one on top of the other.

While the method may be applied to many bones and bone structures, it is especially applicable to bones such as vertebrae, where the plates are inserted through a bore drilled into the body of the vertebra through a pedicle of the vertebra. The bore may be used in other bones as well. Preferably, the diameter of the bore is in the range between 4 to 8 mm.

Preferably, the delivery tool comprises a canula and a rod with which the plates are each advanced through the canula. However other convenient tools, general purpose or specially built may be used, and the selection of the tool is a matter of technical tools dictated by such factors as the surgeon's preference, the specific bone to be mended, or other similar considerations. In some cases, a tool may be further provided, or the same tool used, for holding purposes, e.g. the rod is provided with holding means to hold the plates. Tools may also be used in combination, such as a canula and a rod, and the like.

Furthermore, in accordance with another preferred embodiment of the present invention, there is provided a method for reconstructing and supporting within a diseased or fractured bone or within a space previously occupied by a diseased intervertebral disc the method comprising:

providing a plurality of plates adapted to be separately inserted into the bone and arranged adjacently within the bone or space to construct scaffolding for providing support;

providing delivery means having low profile for delivering each plate through a small incision in the skin of the patient and into the bone or disc;

delivering each plate separately into the bone;

arranging the plates one adjacent the other.

Other aspects and features of the present invention are described in detail hereinafter.

### DETAILED DESCRIPTION

The present invention relates to repair of damaged bones, primarily to damaged or diseased vertebras, and in particular it appeals in relation to compressed fractures of the body of the vertebra caused by trauma or related to osteoporosis. Similarly, although a slightly diferent approach is required, the present invention may relate to fixation of the spine, in cases of degenerative intervertebral disc disease, where the structure disclosed herein may serve as intervertebral fixation device similar to an intervertebral cage.

In accordance with a preferred embodiment, the vertebral reconstruction and support implant method is a minimally invasive surgical method, involving inserting plates, through a small incision in the skin and surrounding muscle tissue, using low profile (i.e. narrow) delivery tools, into the vertebral body or into the inter-vertebral disk area, in order to reconstruct the original anatomical structures. The method fits in particular to collapsed vertebral body or degenerative disk space. After using it for reconstruction of the anatomical structure of the vertebral body, this assembly is used as a prosthesis, which supports the vertebra internally (within the cortex) or externally (intervertebrally), substantially maintaining the normal original shape of the vertebra and the spinal structure.

A typical vertebral modular support implant system comprises a plurality of plates capable of being mounted one on top of the other or next to each other in a lateral adjacent configuration and staying secured in that position so as to present a modular scaffolding structure.

The shape of these plates is designed to allow precise sliding of every plate on top, bellow, or next to the other. In a preferred embodiment of the present invention, in order to accomplish that aim, a recess and corresponding protrusion design is used. It is very desirable that the plate design ensures the prevention or substantial restraining of the plates from sliding off each other.

In order to place each of the plates in the desired position and location a preferable delivery system is used. The characteristics of such system are explained hereinafter.

Insertion and placement of the plates one on top of the other or next to the other creates a wall or stent, that reconstructs and supports the anatomical structure of the organ treated.

The present invention, although not limited to this purpose only, presents a system and method that is particularly suited for treating fractured and compressed bones and more particularly compression fracture of the vertebral bodies. In an alternative embodiment of the present invention it is suggested to implement the modular support implant device for treating a degenerative disc disease, by replacing the diseased disc or most of it and positioning the modular support implant device intervertebrally.

The implementation of the present invention requires minimally invasive surgery that significantly reduces damage to adjacent tissues existing around the treated organ, and is usually much faster to perform, reducing surgical procedure time, hospitalization and recovery time, and saving costs.

An important aspect of the present invention is using a method and device (modular plate construction in our case) to reconstruct anatomical structure, and then use the same device, that changes its role, serving as a reconstructing and fixing device to be left as an implant on location.

The above-mentioned concept brings about several additional advantages and properties that can be characterized as follows:

The present invention introduces a minimally invasive method and approach for treating the affected bone hence causing minimal damage to adjacent tissues and anatomical structures. In addition it uses a prosthesis built from plates to reconstruct a compressed bone back to its normal structure, forming a scaffolding structure to support the vertebral body or other structure treated. This is done while saving essential surrounding ligaments muscles and other tissues responsible for providing stabilization of the vertebral column.

Primarily the purpose of the present invention is to provide a solution for compressed or burst fractured vertebras. The present invention has a real appeal for osteoporosis related compression fractures too. However it is asserted that the present invention may be used to treat degenerative disc diseases by replacing an ill intervertebral disc and enhancing spine fixation.

In a preferred embodiment of the present invention reconstruction of the vertebral body is achieved by bilateral insertion of plates through both pedicles, in two sets, each set arranged one on top of the other, or both sets in an alternating order, to create a double wall-like prosthesis. In other words jacking the collapsed end plates of the vertebra is achieved by gradual expansion of the implant constructed from the inserted plates. In a preferred embodiment of the invention both sets are interconnected at one end to present a corner or a united bond. In another preferred embodiment (for example intervertebral implementation) it may be possible to built more than two scaffoldings (i.e. construct more than two such supporting structures).

Building an implant inside the treated area is a novel concept and technique of treatment, derived from the need to cause minimum damage to tissue while operating on a patient by employing minimally invasive technique. Other operation techniques of vertebral bones require open surgery, hence creating damage to healthy tissue.

Reference is now made to FIG. 1 illustrating an elevated view of two embodiments of a vertebral modular support implant device in accordance with the present invention, implanted in the body of a vertebra.

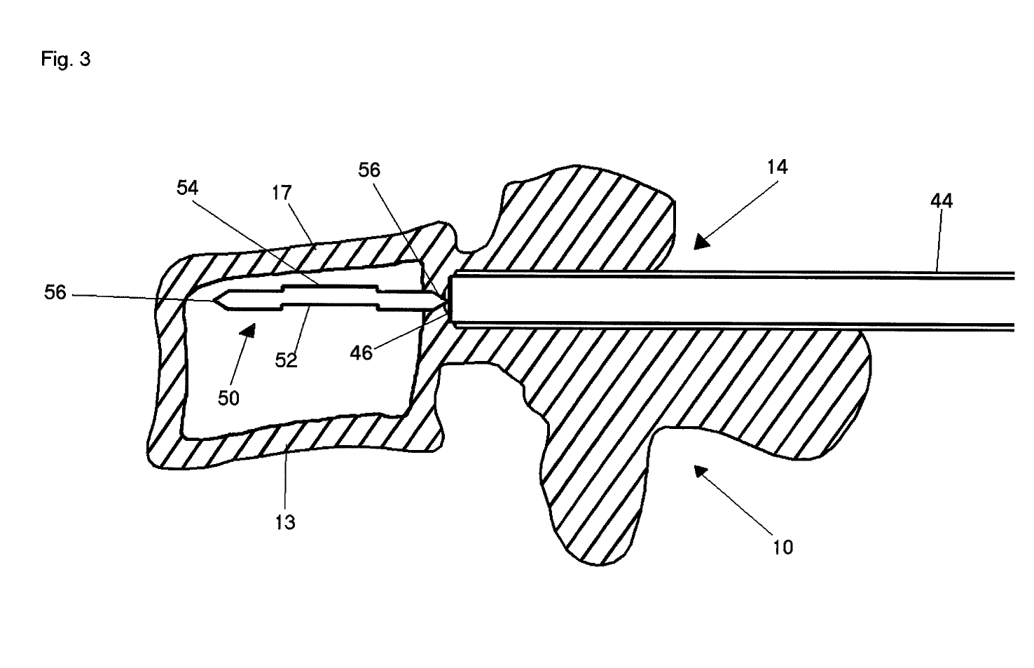

Into the damaged vertebral body 12 at least one vertebral modular implant support device is inserted and erected. In FIG. 1 two such structures are shown—a straight structure 32 and a curved structure 30. A curved structure provides better stabilization although a straight structure may also be considered (or even preferred for various reasons). The vertebral modular implant support structure is made of a plurality of plates, mounted one on top of the other in a desired height in order to provide support for the bone—the body cortical bone (13—see FIGS. 2-5) in the case of a vertebra from within the body. The plates are inserted into the vertebral body via a drilled bore (34 for structure 30 or 36 for structure 32) through the pedicle 14 cortex. Typically the diameter of the bore is anticipated to range between 4 to 8 mm according to the size of the vertebra and its pedicle (but the present invention is not limited to these measurements).

A preferred method of deployment of the vertebral modular implant support device is hereby explained with reference to FIGS. 2 to 5 illustrating various stages of intra-vertebral implant surgical implantation.

The vertebra is accessed in a minimally invasive manner. A guide 42 (see FIG. 2) is inserted through a small incision in the patient's skin and through the muscle tissue towards the vertebra, approaching it in the direction of one of the pedicles. The pedicle 14 is chosen to be the one nearest the desired target position of the vertebral modular implant support assembly. Note that it is recommended to employ the modular implant support device bilaterally, i.e. deploy two such modular constructions through both pedicles. However deployment of the implant support device through only one pedicle is also possible and is covered by the scope of the present invention. The guide is provided with a tapered distal end and is used to puncture and penetrate pedically the vertebra into the vertebral body.

Once the guide is positioned a drill 40, that is advanced over the guide, is used to drill a bore through the pedicle into the vertebral body 12. The external wall 13 of the body (as well as the posterior portion of the vertebra) is made from cortical bone, whereas the inside 11 of the body is cancellous or spongeous, and the bore is extended into the inside of the body. The drill is provided with a lumen extending through it, through which the guide 42 passes reaching the inside of the body.

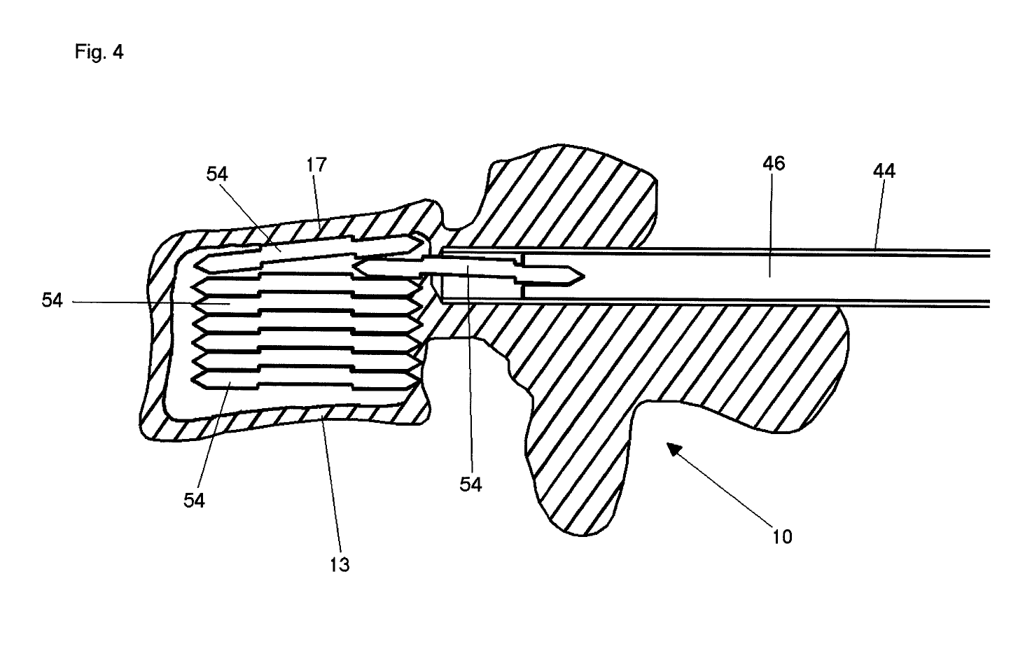

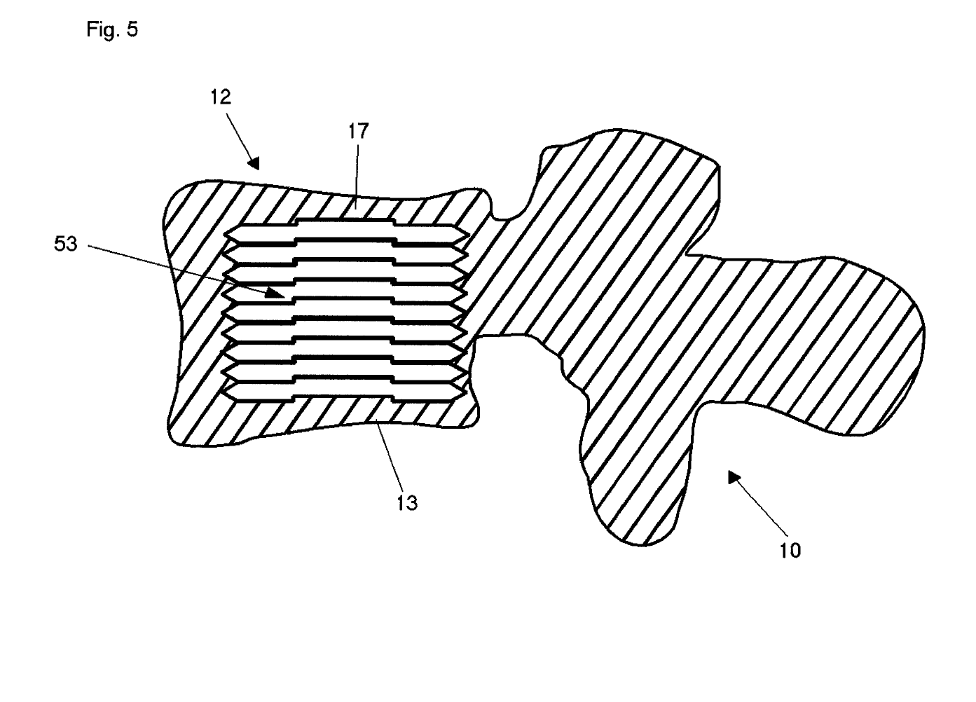

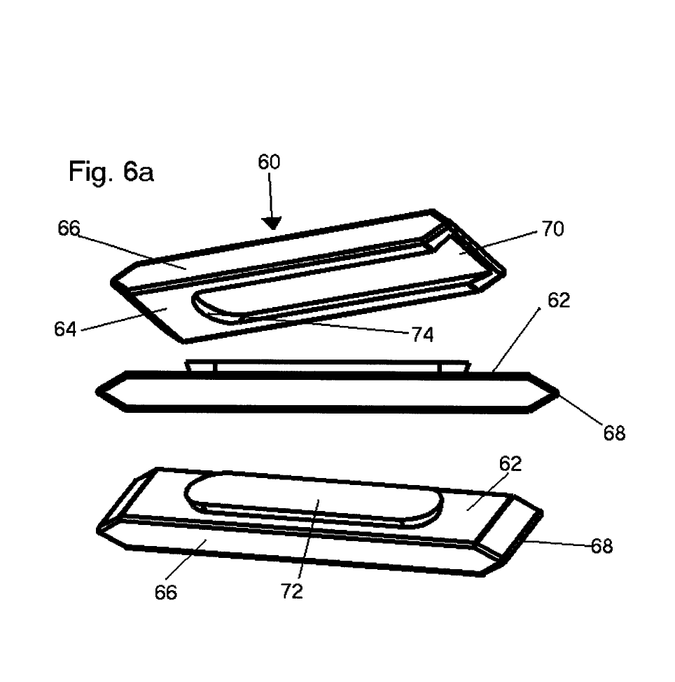

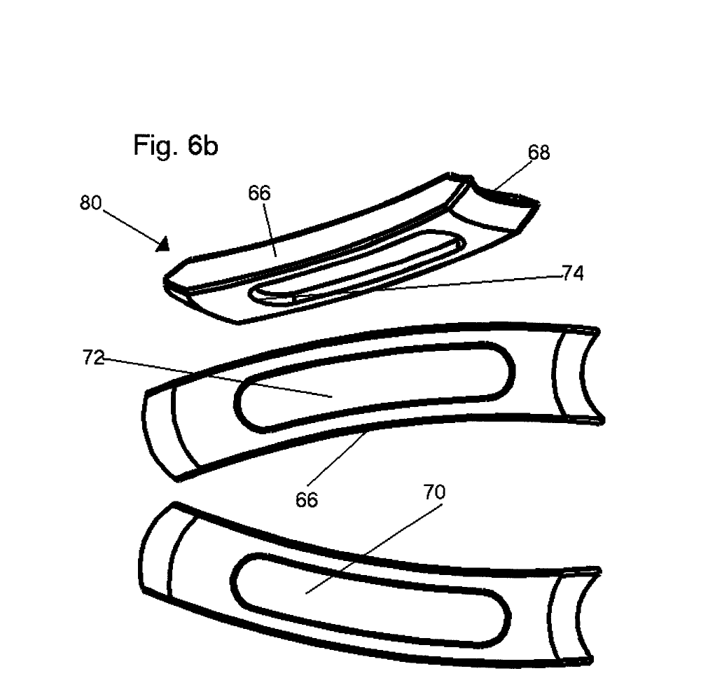

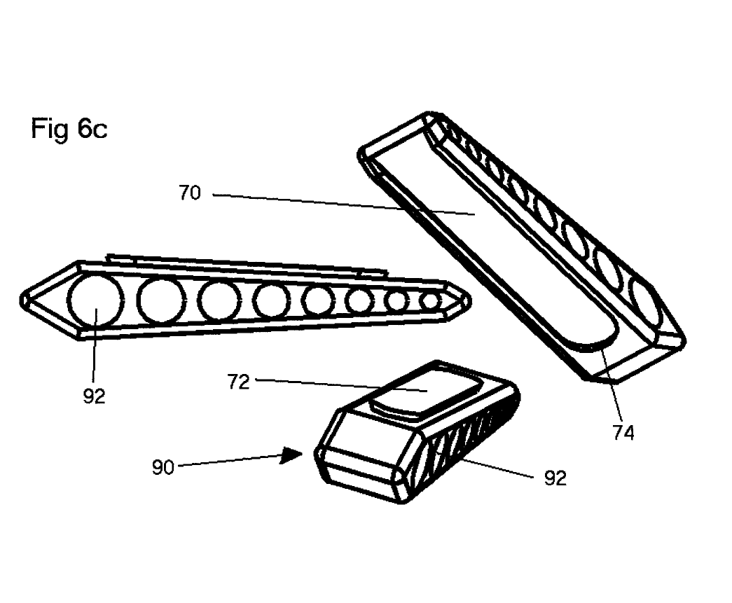

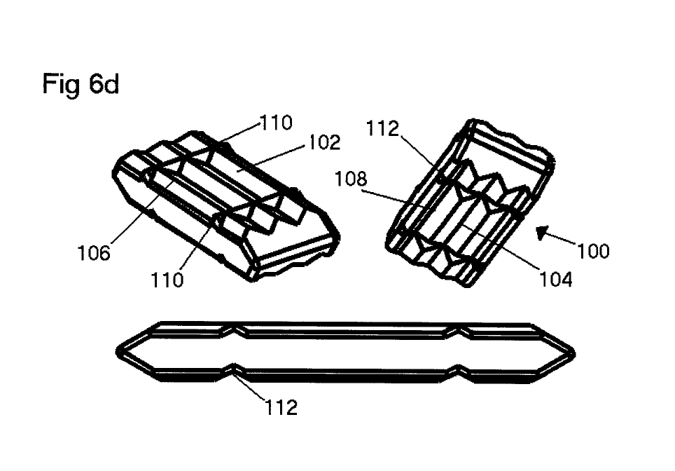

After the bore is drilled, the drill is removed and a canula 44 (see FIG. 3) is guided over the guide 42 through the bore (when in position the guide is removed). Optionally the canula may be provided with external thread for screwing it into the drilled bore and achieving enhanced stability. A first plate 50 is inserted through canula 44, advanced by a delivery tool, which may be a tube, a rod or similar elongated tool, until it is fully inside the body, and positioned in the target location. The delivery tool may include a holding facility at its distal tip for holding the plate and release it on location, or simply push the plate to advance it. The plate 50 is designed to form a building block in a modular structure configuration that is to serve as a support structure within the vertebral body. In one preferred embodiment of the plate in accordance with the present invention, the plate is elongated, having at least one—in this case two—wedged ends 56 so as to allow inserting the plate between adjacent plates (see also FIGS. 4 and 5). The upper surface of the plate is provided with projection 54 that fits into a corresponding recess 52 of an adjacent plate, so as to enhance the stability of the modular structure. Optional design examples are presented in FIG. 6. Preferably, imaging techniques such as fluoroscopy or navigation systems are used in order to facilitate correct positioning of the plates, however other visual or tactile means may be employed.

Similarly, more plates 54 are inserted into the body (see FIG. 4). Note that subsequently inserted plates are guided into position on top (or bottom, or side by side) of the adjacent plate due to the nature of the topography of the adjacent plates, i.e. the indented surface on one plate and the corresponding protrusion of the adjacent plate.

More plates are inserted and guided into the vertebral modular implant support assembly 53 that is formed within the vertebral body 12, until a desired height is reached, facilitating jacking of the vertebral end plates (bottom end plate 13 and top end plate 17) further apart to the original (or new desired) position, preventing the collapse of the wall inwardly. At that stage the delivery tool and the canula are removed. In the natural healing process of the bone the bore is filled with new bone matter, and the vertebral modular implant support assembly is embedded within the bone securing its position and stability.

Note that the present invention may be implemented for providing support to enhance fixation in an intervertebral space previously occupied by a disc. The delivery method may be any minimally invasive approach. Currently there are some minimally invasive approaches for example endoscopic nucleotomy, etc. Such methods may be used, possibly with minor adjustments, in conjunction with the present invention.

FIGS. 6a-6d illustrate several optional configurations for a single plate. Each Figure illustrate three plates of the same sort, viewed from different angle. The plate of the present invention generally comprises a plate having at least two substantially opposite aspects designed to interlock. For the purpose of the present invention “interlocking” means any interlocking mechanism including various types of joining (such as binding, clasping, gripping, interlocking, uniting, hooking etc.), and also partial hooking that merely enhances the stability of the mounted plates.

plate 60 in accordance with a preferred embodiment of the present invention, shown in FIG. 6a comprises an elongated flat plate having two generally opposite aspects—one aspect being the top surface 62 and the opposite aspect being the bottom surface 64 of the plate, and two narrower side aspects 66. The far ends 68 of the plate are wedged (or tapered) so as to allow guiding the plate through and positioning it between two adjacent plates, by separating them apart and sliding therebetween. On the bottom surface 64 a recess 70 is provided, corresponding to a projection 72 on the top surface 62, so as to allow sliding of two adjacent plates—one on top of the other, and preventing their sliding off each other. It is optional to provide a rim 74, either partially, allowing leveled sliding in of the projection of the adjacent plate, as shown in FIG. 6a, or about the entire recess, as shown in FIG. 6b, that serves to retain the projection of the adjacent bottom plate, preventing or at least limiting longitudinal relative displacement between adjacently mounted plates. In the plate shown in FIG. 6b the lateral aspects 66 are mutually curved in a configuration that is aimed at enhanced stability.

The plate 90 in accordance with another preferred embodiment of the present invention, shown in FIG. 6c is aimed at providing inclined support, its top and bottom surfaces inclined with respect to each other rendering one end higher than the other, so that by mounting several plates on top of each other, the total angle of inclination of the vertebral modular implant support assembly is the sum of inclination angles of each of the plates. The plate is provided with a plurality of bores 92 extended laterally across the plate, which may serve for enhancing bone ingrowth and thus enhance incorporation of the implant with the bone structure.

In the plate 100 in accordance with another preferred embodiment of the present invention, shown in FIG. 6d the top surface 102 is provided with longitudinal protrusions 106 (at least one) and optionally two lateral protrusions 110 (at least one), whereas the bottom surface 104 is provided with corresponding longitudinal recesses designed to accommodate the longitudinal protrusions of the adjacent plate, and two lateral recesses 112 designed to accommodate the lateral protrusions of the adjacent plate. This configuration has particular enhanced stability, both in lateral and longitudinal aspects.

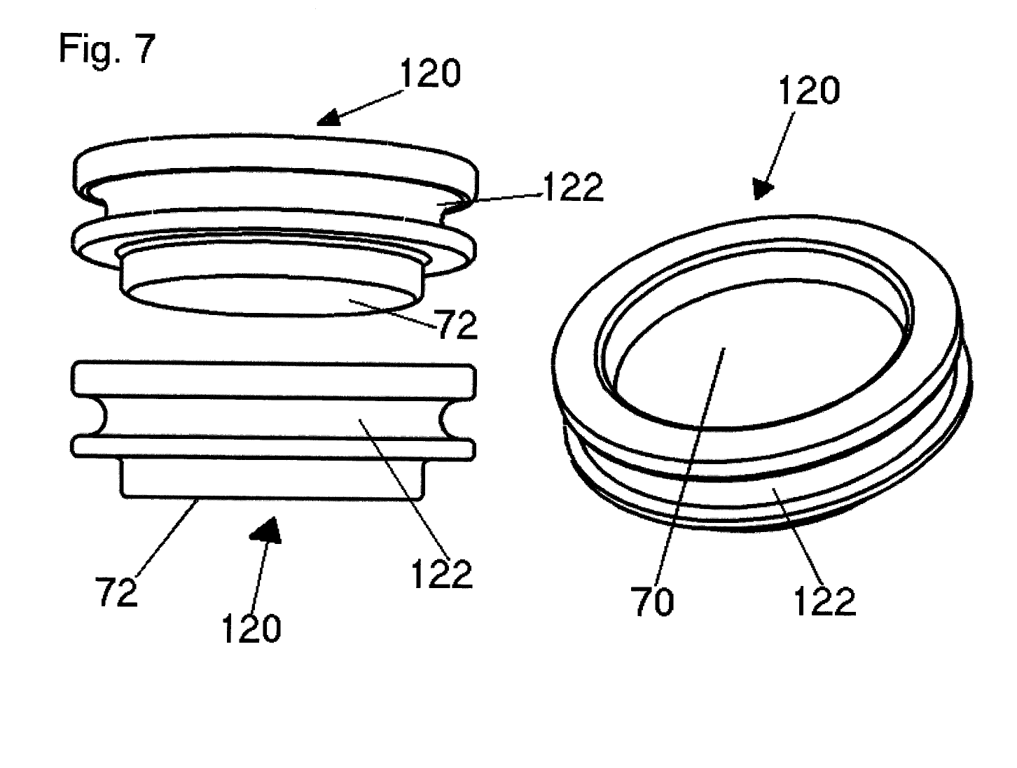

FIG. 7 illustrates yet another alternative embodiment of the plate (showing it in three views), in the form of a disc. The plate 120 is shaped like a disc, with a round protrusion 72 on one aspect (here on the bottom) and a corresponding recess 70 on the other opposite aspect (on top). An optional groove 122 is provided around the lateral aspect of the disk around its perimeter for holding the plate by means of a wire or string, that may be removed or discarded once the plate is in position.

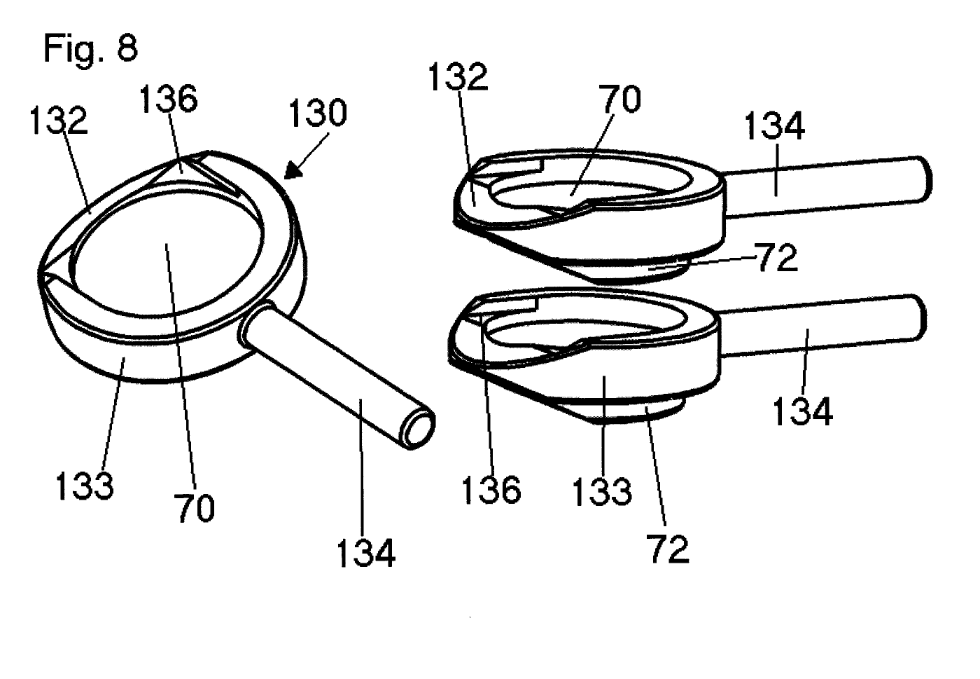

FIG. 8 illustrate still another alternative embodiment of the plate (showing it in three views). The plate 130 consists of two general parts—a disc 133 and a pin 134 coupled to the disc protruding laterally. The pin 134 is provided for holding it (by a delivering tool) so as to ensure its safe guiding to its target position. The disc has a protrusion 72 and an opposite corresponding recess 70 and is tapered 132 on the side opposite to the pin. The protruding pin may protrude in various directions (i.e. not only laterally), provided it is possible to guide it through the guiding canula, or possible to achieve its final positioning by other delivery means.

The plates may be also arranged side by side (with the aspects previosly referred to as “top” or “bottom” in the explanation hereinabove lying side by side laterally), to provide a lateral supporting construction.

By inserting a plurality of plates into the desired position within the bone or space previously occupied by intervertebral disc, it is possible to fill the space substantially with the plates for enhanced fixation.

Again, it is emphasized that these are merely several alternatives suggested. The features of the plates, and in particular the guiding features, may be designed in various ways, and a person skilled in the art could easily design other such guiding features that are different from the features described herein. However the scope of the present invention is not limited to the guiding features described herein in the specification and accompanying figures, but rather defined by the appended Claims and their equivalents. It is also noted that it may be desired to mount plates of various types or shapes on top of each other (for example using several plates shown in FIG. 6a in conjunction with one or several plates shown in FIG. 6c, etc.). Thus this invention further contemplates the creation of plates of various shapes and sizes having compatible locking mechanisms.

The top and bottom aspects may be designed in various shapes and textures (some of which are shown in the drawings.), and it is recommended to provide rough surfaces in order to enhance the friction between the plates and reduce their tendency to slide off each other.

The plates may be provided in various designs, such as straight, laterally curved, different elevations etc., according to the physical features sought. In a preferred embodiment of the present invention it is suggested to build two such vertebral modular implant support assemblies that form two walls with an angle between them, determined by the different pedicular entry angles (see FIG. 1). In another preferred embodiment it is suggested to couple two vertebral modular implant support assemblies at their adjacent ends.

The plates may be made from a rigid biocompatible material, for example metals such as titanium, steel 316, processed foil, hydroxyapatite, or material coated with hydroxyapetite, plastics (polimeric materials), silicon, composite materials (such as carbon-fiber), etc.. The plate may be covered with other substance encouraging bone growth on the implant (such as bone morphogenic protein). In yet another preferred embodiment the plates may be covered with medication substances, such as antibiotics, or slow releasing medication, such as chemotherapic substences, for long-term therapy. If it is desired to implant the vertebral modular implant support assembly in a magnetic resonance imaging (MRI) procedure the plates should be made from non-ferrous materials.

Other coating, such as lubrucants for improved sliding of the plates into their target position, or coating materias that sublime or react to form a solid conglomerate, may be added too. Different coatings may be combined if compatible and beneficial.

It is noted that in particular cases it may be enough to implant only one plate without adding additional plates on top or next to that plate.

Present research contemplates development of materials that will be implantable within a bone and during the course of time give way (dissolve) to bone material. The present invention may be implemented with such materials as well.

The method described herein is minimally invasive and as such has special appeal, for it substantially minimizes surgery-related infection risks, reduces the surgical procedure steps (and thus the costs involved), and shortens healing and recovery times for the patient.

It should be clear that the description of the embodiments and attached Figures set forth in this specification serves only for a better understanding of the invention, without limiting its scope.

It should also be clear that a person skilled in the art, after reading the present specification could make adjustments or amendments to the attached Figures and above described embodiments that would still be covered by the following Claims and their equivalents.

1. A modular reconstructing and supporting assembly for reconstructing and supporting a diseased or fractured bone or within a space previously occupied by a diseased intervertebral disc, the assembly comprising:
a plurality of plates adapted to be cooperatingly inserted into the bone, at least one of said plates arranged adjacently to another plate within said bone or space, to construct scaffolding for forming a supporting prosthesis.

2. The assembly of claim 1, wherein at least one of said plates having at least two substantially opposite aspects with interlocking features designed to facilitate interlocking of adjacent plates so as to prevent or restrain relative movement therebetween.

3. The assembly of claim 2, wherein the opposite aspects of the plate are inclined with respect to each other.

4. The assembly of claim 2, wherein one of said aspects is provided with at least one longitudinal protrusion and the opposite aspect is provided with at least one corresponding longitudinal recess designed to receive a longitudinal protrusion of an adjacent plate.

5. The assembly of claim 2, wherein one aspect is provided with at least one lateral protrusion and the opposite aspect is provided with at least one corresponding lateral recess designed to accommodate a lateral protrusion of an adjacent plate.

6. The assembly of claim 2, wherein one aspect is provided with at least one longitudinal protrusion and at least one lateral protrusion and the opposite aspect is provided with at least one corresponding longitudinal recess designed to accommodate a longitudinal protrusion of an adjacent plate, and with at least one corresponding lateral recess designed to accommodate a lateral protrusion of an adjacent plate.

7. The assembly of claim 2, wherein the interlocking features include at least one recess on one aspect and at least one corresponding projection on the other aspect, so that the projection of one plate is accommodatable in the recess of an adjacent plate.

8. The assembly of claim 7, wherein the recess further comprises a rim adapted for retaining the projection of an adjacent plate, for preventing or restraining relative displacement therebetween.

9. The assembly of claim 8, wherein the rim extends along a portion of the circumference of the recess, allowing leveled sliding in of the projection of the adjacent plate.

10. The assembly of claim 1, wherein at least one of said plurality of plates is curved.

11. The assembly of claim 1, wherein the plate is provided with at least one tapered end, for facilitating plate guidance and positioning between two adjacent plates.

12. The assembly of claim 11, wherein the tapered end is in the form of a wedge.

13. The assembly of claim 1, wherein the plate is made from or coated with biocompatible material.

14. The assembly of claim 1, wherein the plate is made from material selected from a group consisting of metal, titanium, steel 316, processed foil, hydroxyapatite, plastics, silicon, composite materials, carbon-fiber, or a combination thereof.

15. The assembly of claim 1 wherein at least one of said plates is coated with hydroxyapetite

16. The assembly of claim 1, wherein the plate is covered with a bone growth encouraging substance.

17. The assembly of claim 1 wherein said plate being is coated with bone morphogenic protein.

18. The assembly of claim 1, wherein the plate is coated with medication.

19. The assembly of claim 1, wherein said plate is coated with a substance selected from the group consisting of antibiotics, slow releasing medication, chemotherapic substances, or a combination thereof.

20. The assembly of claim 1, wherein the plate comprises non-ferrous material.

21. The assembly of claim 1, wherein the plate is coated with lubricating material to facilitate sliding the plates into a desired position.

22. The assembly of claim 1, wherein the plate is coated with coating materials that sublime or react to form a solid conglomerate.

23. The assembly of claim 1, wherein the plate is substantially disc-shaped.

24. The assembly of claim 23, wherein the plate is provided with a groove adapted to be engaged by a holding tool.

25. The assembly of claim 23, further comprising a pin protruding from at least one of said plates, to facilitate placement of said plate.

26. The assembly of claim 1, wherein at least one of said plates having a rough external surface.

27. A plate for use in conjunction with at least another one of a plurality of other plates in a modular reconstructing and supporting assembly for reconstructing and supporting a diseased or fractured bone or within a space previously occupied by a diseased intervertebral disc of a patient, the plate sized small enough to be suitable for separate insertion into the bone or the space and arrangement with the other plates adjacently to construct scaffolding, so as to provide a supporting prosthesis.

28. A method for reconstructing and supporting within a diseased or fractured bone or within a space previously occupied by a diseased intervertebral disc the method comprising:
inserting a plurality of plates into the bone
arranging said plates adjacent one another, within the bone or
space, to construct a support scaffolding.

29. The method of claim 28 further comprising the steps of delivering each plate separately into the bone using low profile delivery means, through a small incision in the skin of the patient, and arranging adjacent plates on top of each other.

30. The method of claim 28, wherein the bone is a vertebra and the plates are inserted through a bore drilled into the body of the vertebra through a pedicle of the vertebra.

31. The method of claim 30, wherein the diameter of the bore is in a range between 4 to 8 mm.

32. The method of claim 29, wherein the delivery means comprises a canula and a rod with which the plates are each advanced through the canula.

33. The method of claim 32, wherein the rod is provided with holding means to hold the plates.

34. The method of claim 28, wherein at least one of said plates having at least two substantially opposite aspects with interlocking features designed to facilitate interlocking of adjacent plates, for preventing or restraining relative displacement therebetween.

35. The method of claim 34, wherein one aspect is provided with at least one longitudinal protrusion and the opposite aspect is provided with at least one corresponding longitudinal recess designed to accommodate the longitudinal protrusion of an adjacent plate.

36. The method of claim 33, wherein one aspect is provided with at least one lateral protrusion and the opposite aspect is provided with at least one corresponding lateral recess designed to accommodate the lateral protrusion of an adjacent plate.

37. The method of claim 33, wherein one aspect is provided with at least one longitudinal protrusion and at least one lateral protrusion and the opposite aspect is provided with at least one corresponding longitudinal recess designed to accommodate the longitudinal protrusion of an adjacent plate, and with at least one corresponding lateral recess designed to accommodate the lateral protrusion of an adjacent plate.

38. The method of claim 34, wherein the interlocking features include at least one recess on one aspect and at least one corresponding projection on the other aspect, so that the projection of one plate is accommodated in the recess of an adjacent plate.

39. The method of claim 28, wherein at least one of said plurality of plates is provided with at least one tapered end, to facilitate positioning the plate between two adjacent plates.

40. The method of claim 28, wherein at least one of said plurality of plates is substantially disc-shaped.

41. The method of claim 28, wherein at least one of said plurality of plates is further provided with a protruding pin, adapted to facilitate holding the plate by a delivering tool.

42. The method of claim 30, wherein the plates are inserted bilaterally constructing at least two scaffolding structures within a vertebral body.

43. The method of claim 28, wherein the plates are positioned one on top of the other.

44. A method for reconstructing and supporting within a diseased or fractured bone or within a space previously occupied by a diseased intervertebral disc the method comprising:
providing a plurality of plates adapted to be separately inserted into the bone and arranged adjacently within the bone or space to construct scaffolding for providing support;
providing delivery means having low profile for delivering each plate through a small incision in the skin of the patient and into the bone or disc;
delivering each plate separately into the bone;
arranging the plates one adjacent the other.
